(19) United States
(12) Patent Application Publication
(10) Pub. No.: US 2007/0232954 A1
(43) Pub. Date: Oct. 4, 2007

(54) Automated skin biopsy device

(76) Inventors: Jeffrey Paul Harris, La Jolla, CA (US); George Kinyon Houghton, San Diego, CA (US); Adam Benjamin Harris, San Francisco, CA (US)

Correspondence Address: John R. Ross, III, P.O. Box 2138, Del Mar, CA, 92014, US

(21) Appl. No.: 11/397,775

(22) Filed: Apr. 4, 2006

(51) Int. Cl. A61B 10/00; A61B 17/32; A61B 17/08

(52) U.S. Cl. 600/564; 600/568; 606/167; 606/219

(57) ABSTRACT

An automatic skin biopsy device that includes a drive unit and a variety of shapes and sizes of disposable cartridges. The device is capable of automatic extraction of a small and precisely located portion of skin having a shape similar to a wide canoe. A disposable cartridge is mounted onto and driven by the drive unit which incorporates at least one gear motor. In a preferred embodiment the gear motor in the drive unit drives, via gears and cams, two cutter blades in the disposable cartridge through separate curved paths to extract from the skin the generally wide canoe-shaped skin sample. Preferably another gear motor via gear and cam action produces back and forth oscillation of the blades to assure easy slicing of the skin tissue. The canoe-shape incision that is about 1 to 4 mm deep produces a good biopsy sample and the incision is easily closed with a medical staple or sutures. In preferred embodiments a stapler is provided as a part of the sample acquisition device. Materials used to fabricate the driver and the cartridge preferably transparent to permit accurate placement of the incision.

This invention is related medical devices and in particular to automated skin biopsy devices.

### BACKGROUND OF THE INVENTION

A typical prior art method of obtaining a skin biopsy sample is to use a scalpel to incise a skin section in the general shape of an ellipse down through the epidermis and dermis Forceps are used to lift up the elliptical skin section and a cut is made under it to complete the excision. The practitioner then inspects the wound for completeness of the excision, cauterizes it for the control of bleeding if necessary and then sutures the incision site closed with one of various types of suture material or he may use a staple gun to close the incision site. Alternatively, the practitioner may employ punch biopsy devices such as Sterile Disposable Biopsy Punches, Model 33-35, that are available from MedicalMailOrder.Com with offices in Bangor, Me. These devices allow the practitioner to use a circular hollow shaped blade that cuts a circular hole in the skin by using a twisting motion of the hand. The biopsy is then lifted out and a scalpel is used to cut underneath the circular-shaped incision to free it from the subcutaneous tissues. Again after cautery the incision is separately closed.

The cosmetic results from these techniques depend upon the surgical training of the practitioner and the strict adherence to plastic surgery skin handling and suture techniques which are taught during surgical residency training. However, the vast majority of biopsies are in fact done by family practice physicians, dermatologists and internists whose abilities to perform surgery vary considerably.

The time that a biopsy takes with conventional techniques requires a surgical set-up that includes a sterile drape, a sterile forceps, scissors and scalpel and suture package. Quite often this requires a medical assistant or a nurse to participate in the procedure, package the biopsy, send it to a pathology laboratory and re-sterilize the instruments in order to prepare for another biopsy during the day.

At present there are approximately 91,000 skin biopsies done in the U.S. on a daily basis, with about 13,000 registered dermatologists each averaging seven per day. Additional biopsies are performed by other medical entities. The need for biopsies will certainly increase as sun-exposure continues to be the main cause for the development of skin cancers of various varieties (squamous cell and basal cell carcinomas and malignant melanomas) and the fact that more people are living longer so that the consequences of sun-damaged skin are being realized.

What is needed is a device to automatically collect skin biopsy samples in a quick, simple, consistent manner.

### SUMMARY OF THE INVENTION

The present invention provides an automatic skin biopsy device that includes a drive unit and a variety of available disposable cartridges that have different blade dimensions to suit the application . The device is capable of automatic extraction of a small and precisely located portion of skin having a shape similar to a wide canoe or ellipse. A disposable cartridge is mounted onto and driven by the drive unit which incorporates at least one gear motor. In a preferred embodiment the gear motor in the drive unit drives, via gears and cams, two cutter blades in the disposable cartridge through separate curved paths to extract from the skin the generally ellipse-shaped skin sample. Preferably another gear motor via gear and cam action produces back and forth oscillation of the blades to assure easy slicing of the skin tissue. The ellipse-shape incision that is about 1 to 4 mm deep produces a good biopsy sample and the incision is easily closed with a medical staple or sutures. In preferred embodiments a stapler is provided as a part of the sample acquisition device. Materials used to fabricate the driver and the cartridge preferably transparent to permit accurate placement of the incision.

Preferred embodiments are hand held and battery powered. With a press of a button the first motor initiates motion of the curved blades contained in the disposable sterile cartridge so that the blades while oscillating back and forth move first downward into the skin then inward toward each other to under cut the biopsy sample so that the sample is freed from the body and is contained within the cartridge. The cartridge, with the biopsy tissue safely enclosed within it, can then be ejected into a pathology specimen container either with formalin or without depending upon the requirements of the pathologist and practitioner. The practitioner may then cauterize the wound as needed and either use the attached staple gun to quickly close the skin edges or employ routine suturing techniques depending upon his/her preference. Preferred embodiments include large numbers of single use cartridges with several differing blade lengths, cut widths and depths, and curve shapes to accommodate larger lesions or requirements to extract samples of various sizes and shapes. There are instances when complete excision of a lesion is required and alternatively there are times when an incisional biopsy is preferred; thus having the option of different blade dimensions and configurations meets both of these requirements. Important advantages of the present invention is that it saves time, provides a biopsy result superior to punch biopsies and cosmetic results generally equivalent to those obtained by a well-trained and experienced surgeon. This entire procedure takes less than three seconds to complete the entire excision.

### BRIEF DESCRIPTION OF THE DRAWINGS

FIG. 1 is a overall illustration of a preferred embodiment of the present invention.

FIGS. 2A through 4 show drive mechanism details.

FIGS. 5 through 9F show sample cartridge details.

FIGS. 10A, 10B and 10C show a cut pattern in skin produced by a preferred embodiment.

FIG. 11 shows a stapling configuration.

FIGS. 12A and 12B show the sample cartridge and its package

FIG. 13A through 16 and FIGS. 18, 19A and B show details of stapler and its operation.

FIG. 17A through 17E represent a moving picture of a blade actuation.

### DETAILED DESCRIPTION OF PREFERRED EMBODIMENTS

### First Preferred Embodiment

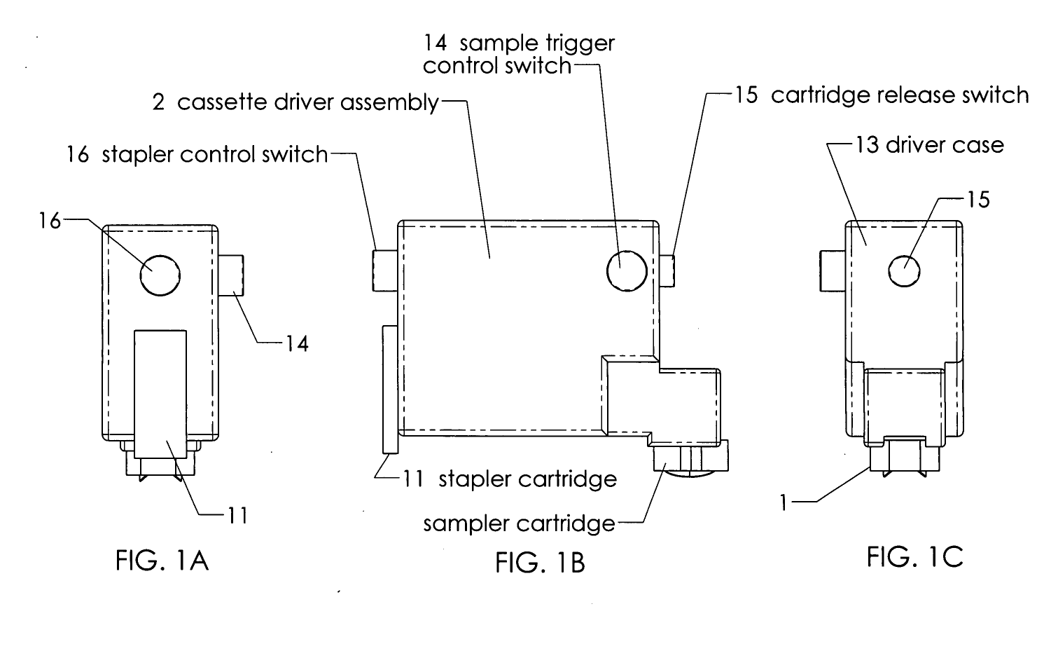

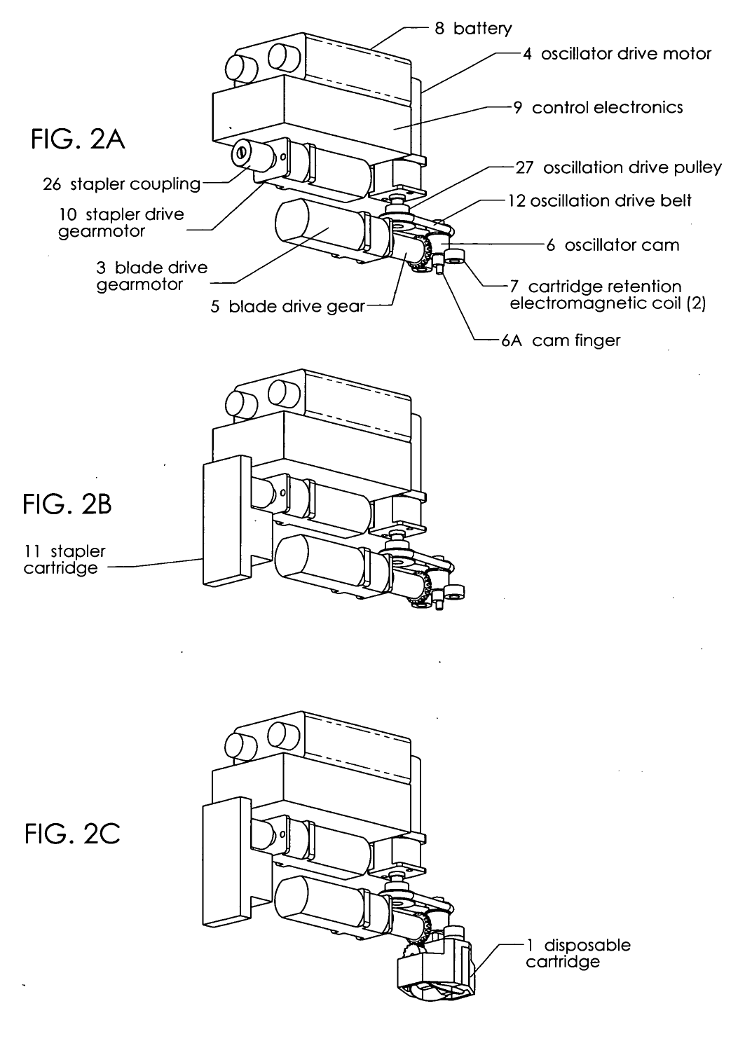

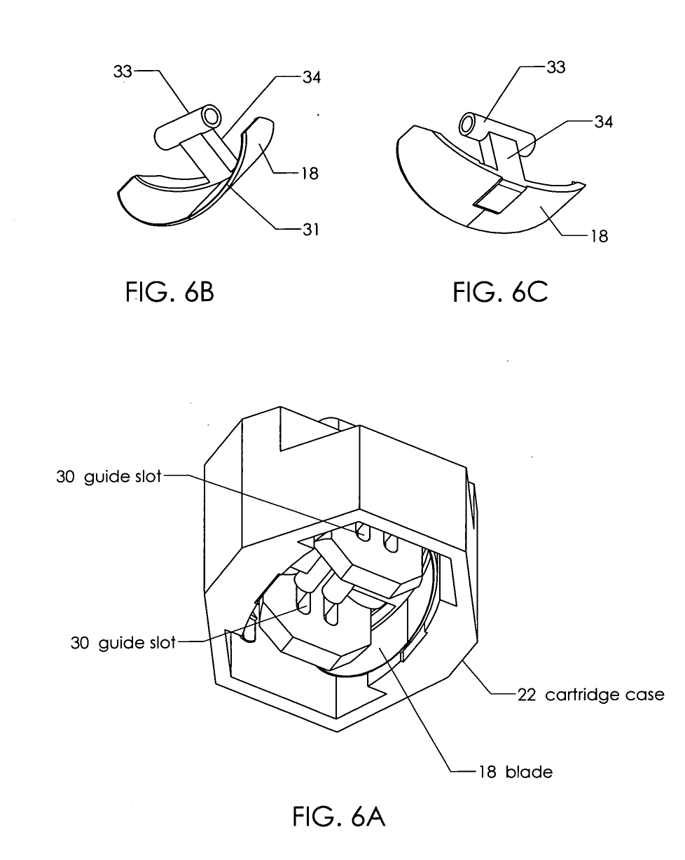

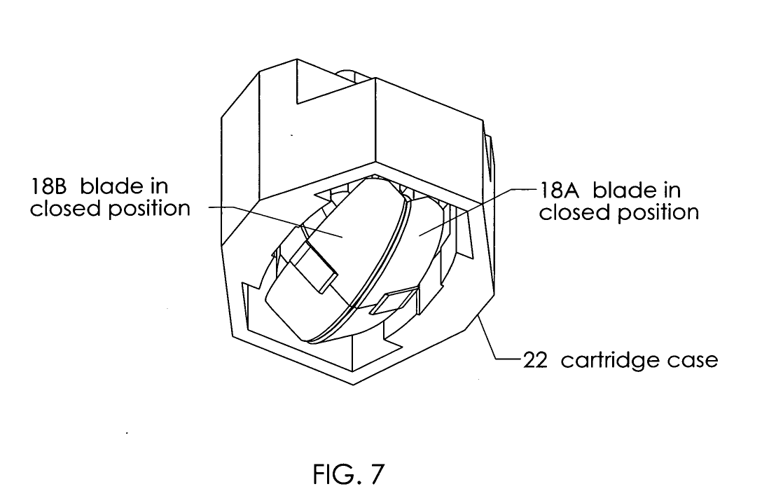

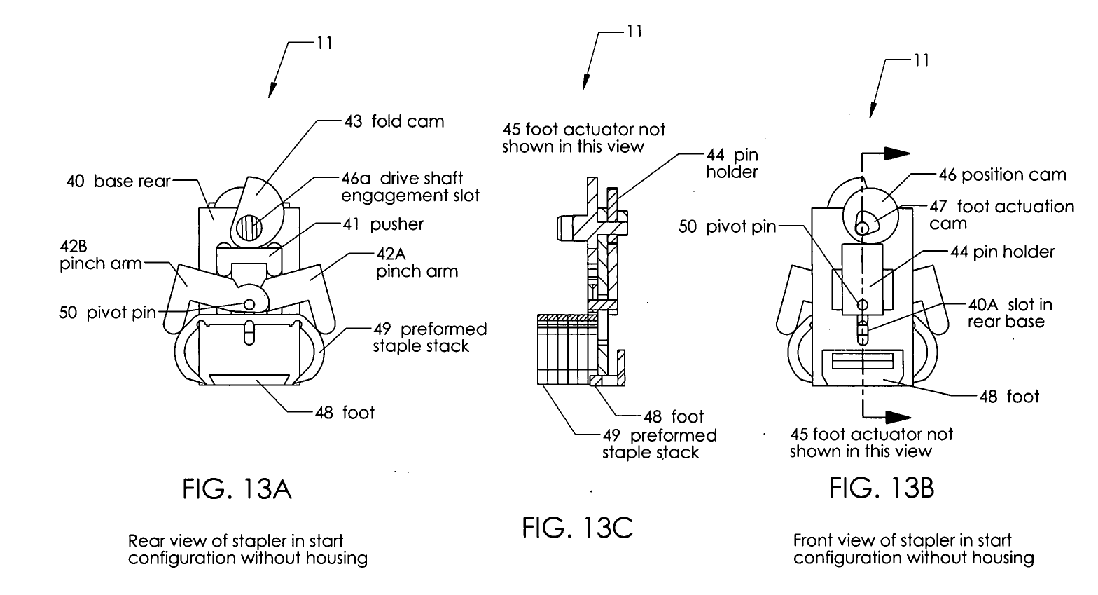

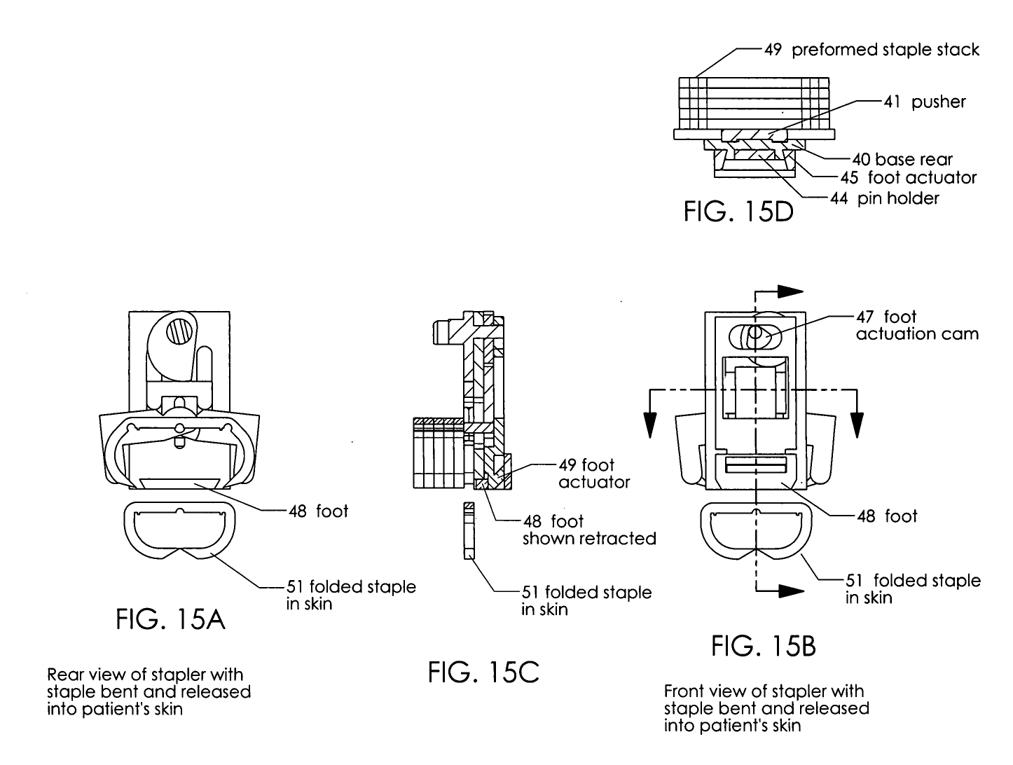

A preferred biopsy assembly of the present invention is shown in FIGS. 1A, 1B, 1C and FIG. 2A, 2B, and 2C. It includes: (1) a drive unit 2 shown in FIG. 2A, (2) a large number of disposable biopsy cartridge assemblies 1, one of which is shown in detail in FIGS. 6 through 9F and (3) a stapler unit shown at 11 shown in FIG. 2D and shown in detail in FIGS. 13A through 16.

Drive Unit

The preferred drive unit 2 includes electronic controls 9, battery unit 8, blade drive gear motor 3, drive gear 5, stapler drive gear motor 10, oscillator drive motor 4, oscillator drive pulley 27, oscillator drive belt 12 and drive oscillator cam 6 with cam finger 6A.

Disposable Biopsy Cartridge Assembly

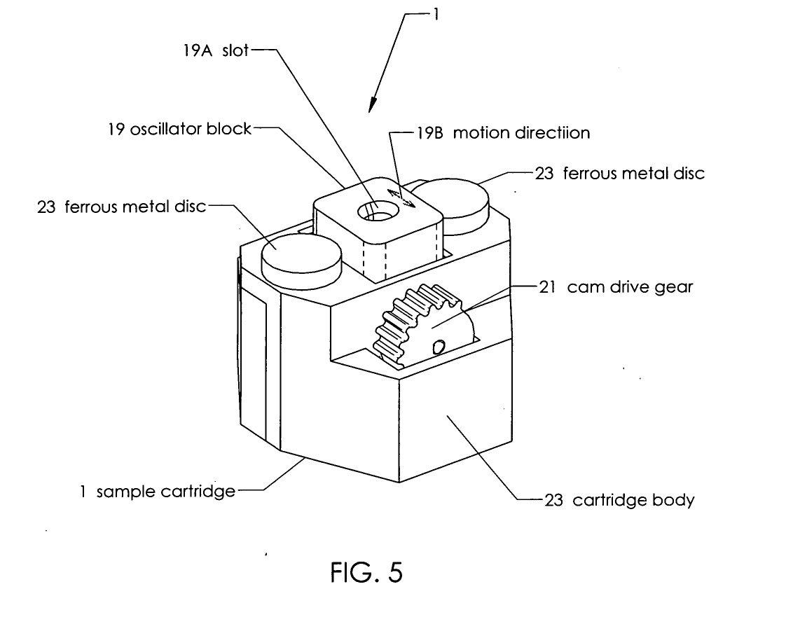

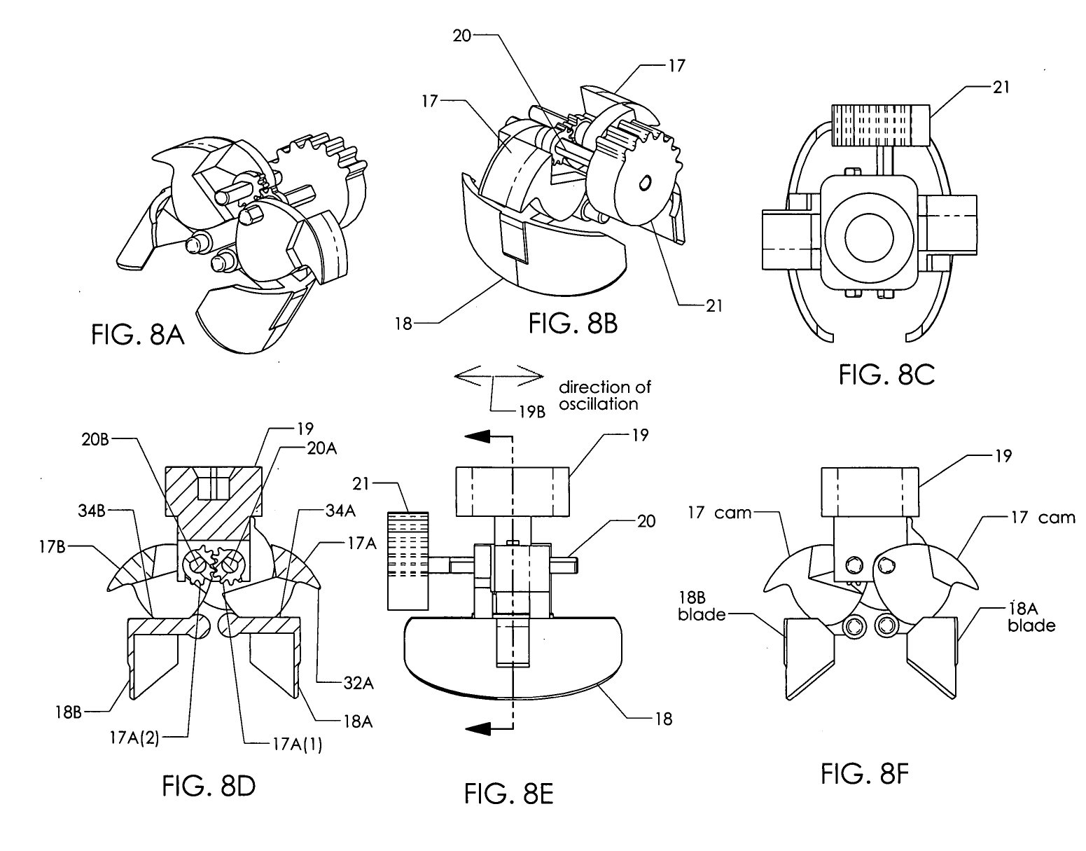

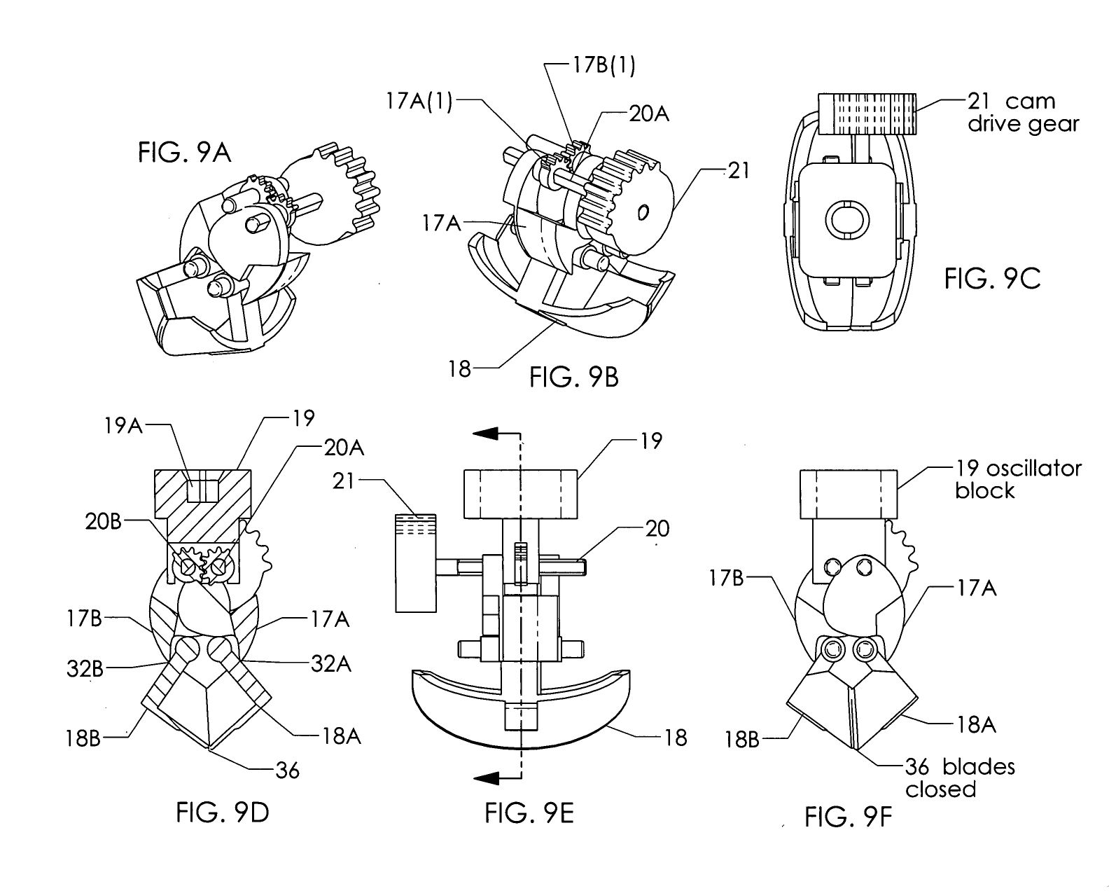

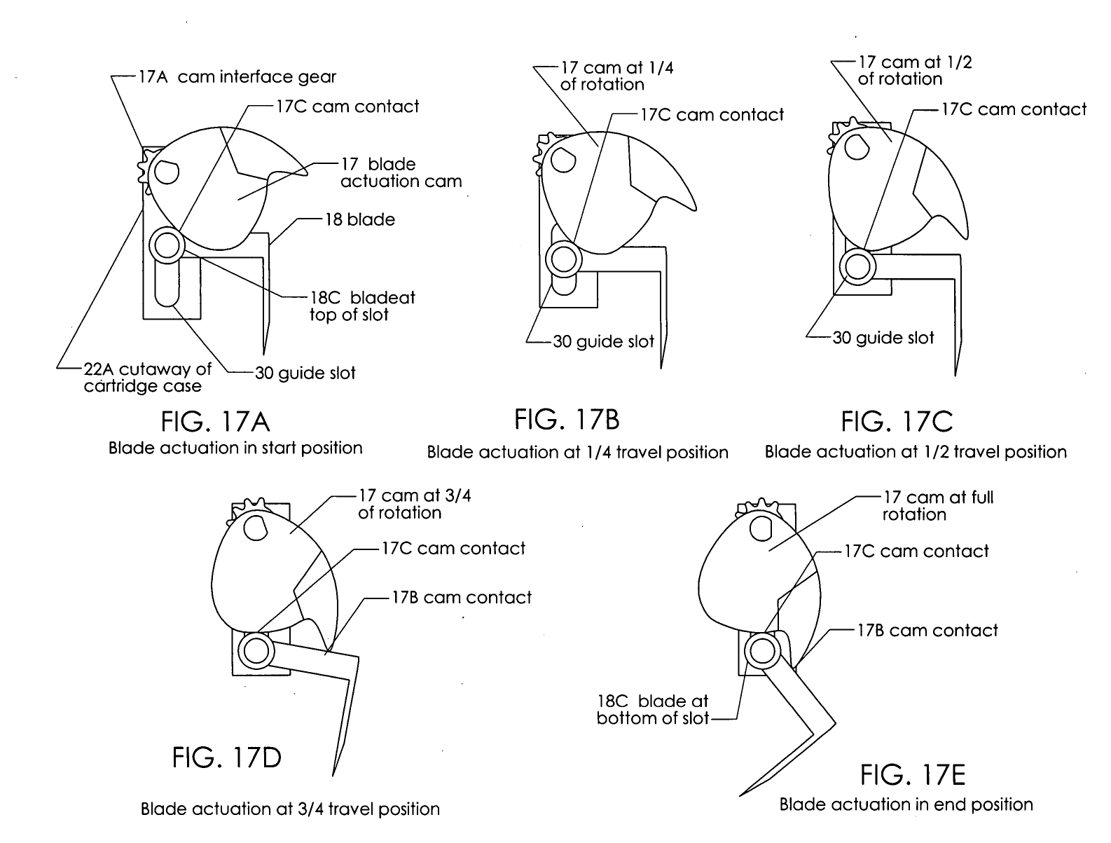

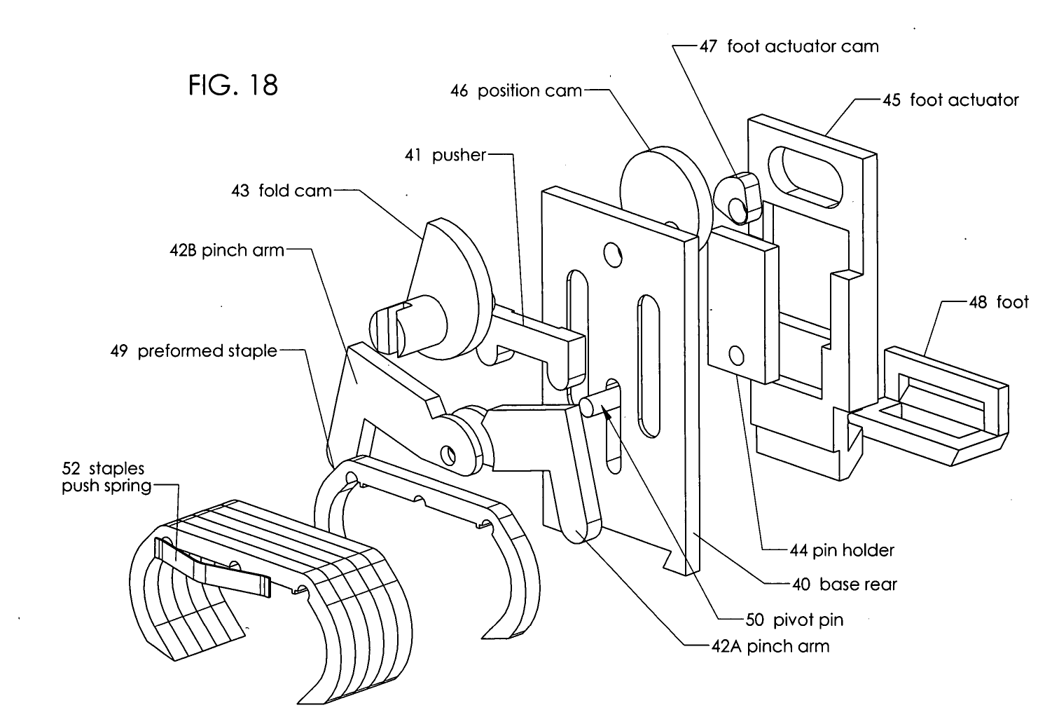

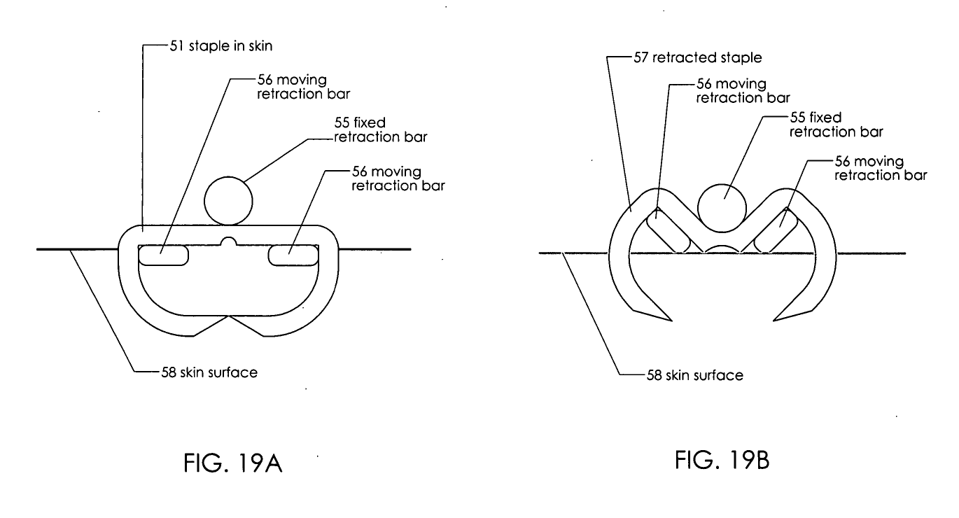

Preferred disposable biopsy cartridge assembly 1 shown in FIG. 5 includes cam drive gear 21 driven by drive gear 5 in drive unit 2. The cartridge also includes oscillator block 19 with finger slot 19A into which cam finger 6A fits and produces a back and forth oscillation of block 19 and blades 18A and 18B as well as all of the other components shown in FIG. 8E. The direction of the back and forth oscillation is indicated by double arrow 19B in FIG. 8E. In a preferred embodiment the extent of the back and forth oscillation is about 0.2 mm. The turning of cam drive gear 21 in cartridge 1 by blade drive unit 2 causes blades 18A and 18B as shown in FIG. 9B to first move in a downward direction slicing into the skin with the back and forth oscillation produced by oscillator cam 6. After the blades have cut into the skin to a predetermined depth within the range of about 2 to 4 mm, the continued rotation of gear 21 and cam action of cam 17A and 17B causes blade 18A and 18B to cut inward toward each other to cut under the skin section and to trap the biopsy sample within the blades as they move to their closed position. Five sequential positions of one of the blade and other cartridge components are shown in FIGS. 17A through 17E.

Stapler Unit

The first preferred embodiment of the present invention also includes stapler unit 10 as shown in FIG. 2B and 2C and in detail in FIG. 13, 14, 15, 16 and 18. The stapler cartridge 11 snaps into the drive unit 2, and a tab in the end of stapler coupling 26 as shown in FIG. 2A engages stapler shaft engagement slot 46A as shown in FIG. 13A. Stapler coupling 26 couples the stapler shaft to stapler drive motor 10 in drive assembly 2. The stapler is used to close the wound after the sample has been extracted.

### Mechanical Operation of the Device

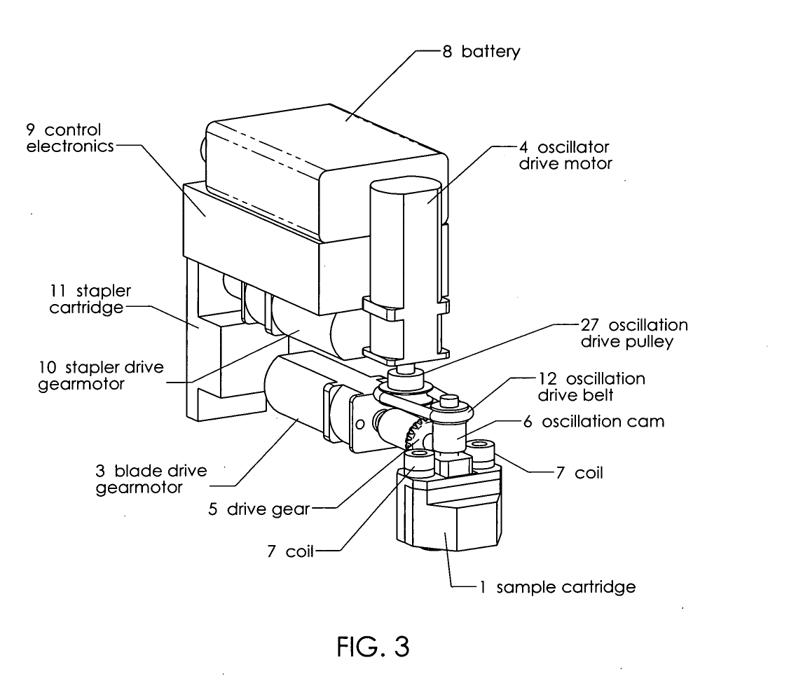

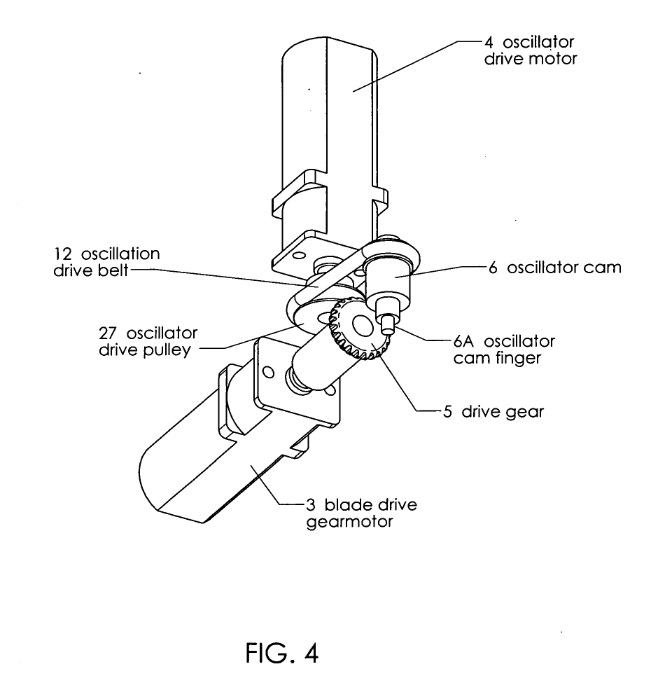

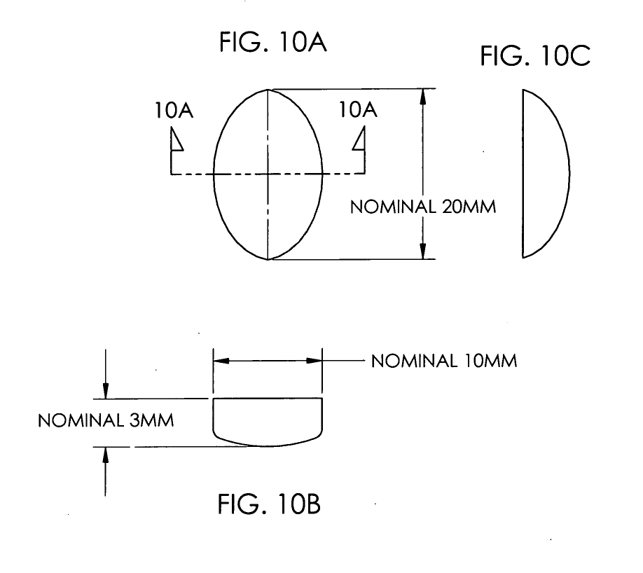

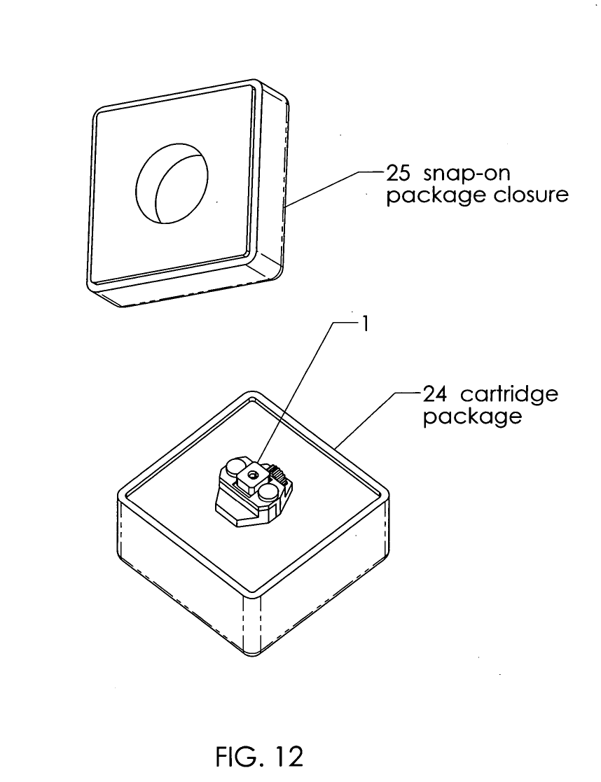

A sterile cartridge assembly 1 is removed from its package 24 as shown in FIG. 12, under sterile conditions and inserted into the driver assembly 2 as shown in FIGS. 1A, 1B and 10. The insertion engages gear 5 in drive unit 2 as shown in FIG. 3 with the cam drive gear 21 in cartridge 1 as shown in FIG. 5. Drive unit 2 comprises electromagnetic coils 7 as shown in FIG. 3. Control electronics 9 are configured such that the pressing the cartridge release 15 once will activate the cartridge retaining electromagnetic coils 7 which will hold the cartridge in its place in the drive 2 by attracting the ferrous metal discs 26 on the sample cartridge 1 as seen in FIG. 5. Cartridge assembly 1 is located, by a medical operator who is intending to obtain a skin sample, over a previously prepared skin location (such as one containing a lesion) from which a sample is required. When the operator presses the control switch 14 shown in FIG. 1 on the driver assembly 2 the control electronics 9 as shown in FIG. 2 receives a start signal, causing the gear motor 3 and oscillation gear motor 4 to begin to rotate their rotors. The output shaft of gear motor 3 is attached drive gear 5 and all begin to rotate with the rotor of gear motor 3. Drive gear 5 is engaged with the cam drive gear 21 in cartridge 1 as shown in FIG. 5 when cartridge 1 is attached to the driver assembly as explained above. As cam drive gear 21 begins to turn, it rotates its attached cam drive shaft 20 as shown in FIG. 8B and blade actuation cam 17A and also partial gear 17A(1) which is an integral part of cam 17A as shown in FIG. 8B. Gear teeth on partial gear 17A(1) are meshed with teeth on a second partial gear 17B(1) on cam 17B to rotate the two cams at the same speed in the opposite direction. As the cams rotate, they begin to force the two identical blades 18A and 18B down toward the skin surface, guided by slots 30 in the cartridge 1. At this time the blades are approximately perpendicular to the skin surface as shown in FIG. 9B and FIG. 17A. At the time the blade drive gear motor 3 begins to turn, oscillation drive gearmotor 4 as shown in FIG. 3, with its attached oscillation drive pulley 27 starts to rotate. Pulley 27 rotates the oscillation drive belt 12 which rotates the oscillation cam 6. Finger 6A shown in FIG. 4 on oscillation drive cam 6 is engaged in slot 19A in the oscillation block 19 in the cartridge assembly 1 at the time the cartridge is attached to the driver assembly. The oscillation cam rotates, at a speed of 200 to 600 rpm in the preferred embodiment, causing the entire interior component set of the cartridge assembly 1 shown in FIGS. 8A through 8F to oscillate back and forth a distance of about 0.1 to 0.5 mm (preferably about 0.2 mm). The back and forth oscillation is preferably in a direction indicated at 19B in FIGS. 8E along the long axis of the proposed canoe shape cut to impart a rapid short slicing action to the blades. As the blades come in contact with the skin surface, they will begin to slice into the skin normal to the surface as shown in FIG. 17A, 17B and 17C. When the blades reach to bottom of their guide slots 30, as shown in FIG. 17C, a second surface on each cam will come into contact with mounting arms 34A and 34B on the blades as shown in FIG. 9D, forcing the blades to start to rotate toward each other as shown at 36 in FIG. 17D. The cut is completed when the blades come together and slightly overlap, as shown in FIG. 9D and as indicated in FIG. 17E cutting the sample free. The sample is thus trapped inside the cartridge, requiring no operator contact. A detent (not shown) on each blade control cam will act to hold the blades in the closed position so as to positively retain the tissue sample. The cut will have produced a skin sample with a cross section similar to the shape of a canoe section and the cut shape and approximate dimensions as shown in FIGS. 10A, 10B and 10C with dimensions of width, depth, and length being defined by the specific cartridge selected. In the preferred embodiments a variety of cartridges are provided with cut depth preferably ranging from about 2 mm to about 4 mm.

### Electronic Control

All the power and timing come via the control electronics module 9 shown in FIG. 2, which is connected to battery 8 and a wire harness not shown. Sensors are provided to mark the rotational position of the drive gear 5 and the presence of the cartridge 1. Cartridge case 15 is provided with internal walls to protect the drive assembly from possible blood contamination. The interior of the drive unit is further protected by flexible seals (not shown) at the motor shafts. The drive assembly is designed for easy cleaning as it is not a disposable item. Should blood contamination occur, the cavity in the driver assembly 2 wherein the cartridge is installed may be rinsed out by any appropriate means using denatured alcohol, betadine, distilled water or the unit may be gas-sterilized.

### Detailed Description of Stapler Unit

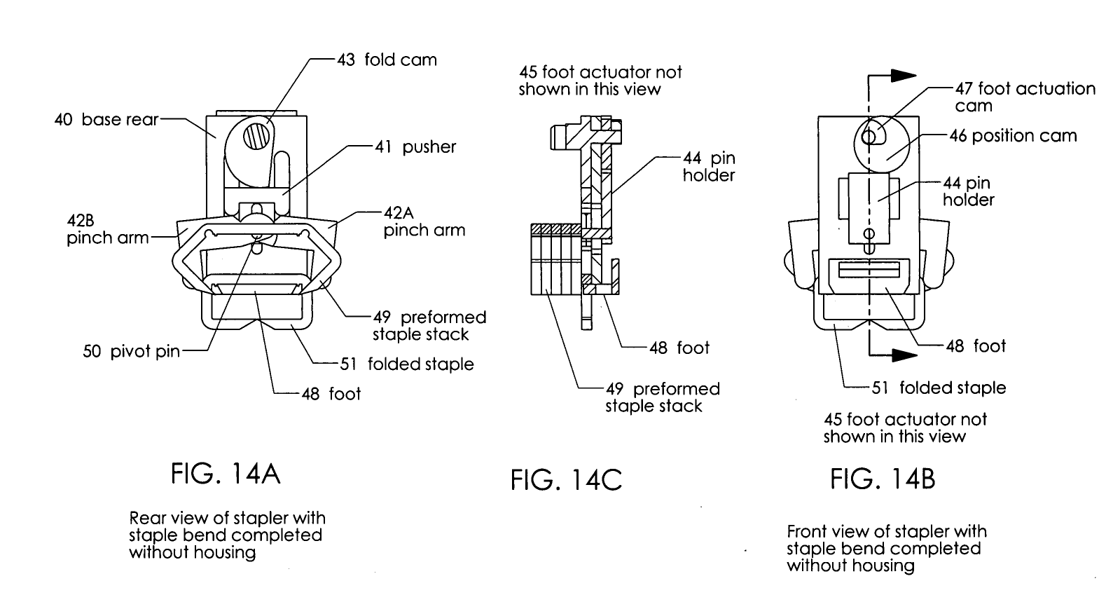

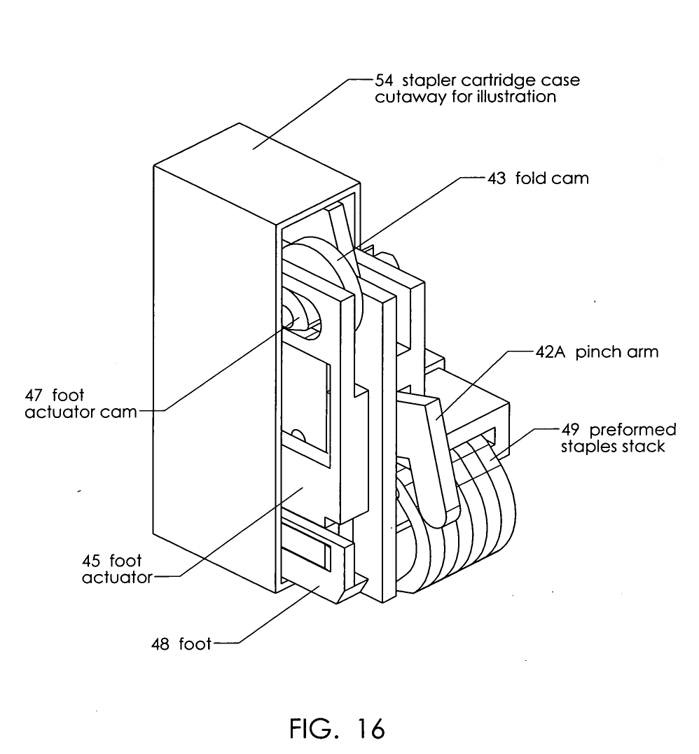

Stapler unit 11 is shown in FIG. 2B and 2C and in detail in FIG. 13, 14 15 and 16. Drive shaft 46 is integral with the fold cam 43, position cam 46, and foot actuation cam 47. The points of the first-in-line staple in preformed stapler stack 49 are positioned typically about 2 mm from each edge of the gap in the skin left by the removal of the biopsy sample. Pressing the stapler control switch 16 in FIG. 1B will start stapler drive gear-motor 10 turning, which turns the cams and initiates stapler operation. As seen in FIG. 13A, 13B, and 13C, when the cams rotate the fold cam 43 contacts pusher 41 which slides in a slot in base rear 40. Tabs on pusher 41 contact the two pinch arms, pusher arms 42A and 42B. At the same time position cam 46 pushes pin holder 44 and its integral pin 50 down toward the bottom of the stapler, carrying the pinch arms 42A and 42B along with it. Pusher arms 42A and 42B pivot around pin 50. The pinch arms 42A and 42B contact the first-in-line preformed staple 49 and push the staple 49 out of stapler case 54 as shown in FIG. 16 and into the patient's skin. Staple 49 is spread in its initial form to allow skin entry outside of the open skin incision. As the staple is folded to the shape shown in FIG. 14A and further skin penetration occurs, the staple pulls of the edges of the incision together to close the wound left by the removal of the biopsy sample.

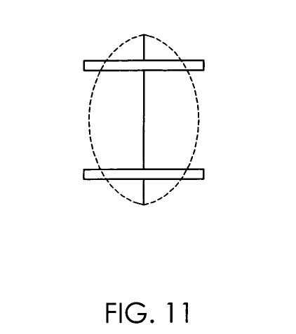

The staple 49 is pushed into the skin until it contacts foot 48, which stops the progress of the staple 49. The pinch arms 42A and 42B continue to rotate, bending the staple 49 to the folded staple configuration 51. The staples 49 may be pre-scored to ease the folding operation. When the folding action is completed continued turning of the foot actuation cam 47 pulls the foot actuator 45 up forcing a tapered tang at the bottom of the foot actuator 45 to work on a similar tapered surface on the foot 48 to pull the foot 48 out from the folded staple 51, allowing the stapler to be removed from the patient with the staple in place as shown in FIG. 11. As the cams continue to turn the foot 48 is pushed back into the start position through action of the foot actuation cam 47 and the foot actuator 45. The other components return to their start positions under the influence of return springs (not shown). A spring in the cartridge (not shown) acts upon the stack of staples 49 to push the next staple into the ready position.

The stapler cartridge 11 is conceived as a single-patient disposable unit, with inexpensive, mostly molded plastic component and a small number of staples to minimize opportunities for contamination of other patients and medical personnel. After use on its patient the stapler cartridge 11 may be snapped out of the drive assembly 2 and discarded in a suitable sanitary receptacle.

Staples may be removed from the patient as shown via use of a tool having a three-pronged configuration as shown in FIG. 19A and 19B. The shape of the staples and their removal tool will minimize tearing of the patient's tissue at removal.

### General Description of Excision Operation

The forward portion of the body of cartridge 1 is preferably molded of clear plastic and is transparent so the operator can see clearly through it to achieve a precise location for the incision. The sample shape as shown in FIGS. 10, 10A and 10B is similar to the plan view of a canoe, with dimensions similar to those shown in the figures. Other patterns can be obtained depending on the cartridge selected. The operator presses the cartridge lightly and evenly against the skin and presses the sample trigger control switch 14. The sample is taken in 1 to 3 seconds. It is trapped inside the cartridge at the end of the cycle. The operator then lifts the sampler away from the skin with the sample trapped in the cartridge. The wound shape is ideal for best closure, most effective healing, and minimal scarring. Using a stapler 10 that in the preferred embodiment is incorporated in the driver 2 as shown in FIG. 1, the operator closes the wound. FIG. 11 shows the preferred position of two staples to close the wound. The operator places the cartridge back into its package 24, which has been maintained in a sterile condition, and presses the cartridge release switch 15. The cartridge drops back into its sterile seat in the package and the package is closed and sealed with closure 28. The package may then be stored or transferred to a laboratory for analysis. Alternatively, the cartridge may be ejected directly into a pathology specimen container filled with a tissue fixative, such as formalin.

### Removal of Sample and Wound Closure

When the incision has been completed and the sample is trapped inside the cartridge the sampling system is lifted from the skin. The cartridge may be placed back inside its sterile package or in some other suitable container at the discretion of the operator by pressing the cartridge release switch 15 to shut off power to the cassette retention electromagnets 7 to allow the cartridge to release from the driver assembly. The operator may then close the wound using the stapler 11 built into the driver assembly, a separate stapler, stitches, or other methods as determined by the operator.

### Clean-Up

The cartridges and their packaging are expected to be single-use, disposable items. The cartridge package 24 is preferably equipped with cartridge locating and seating package base and a snap-on package closure 25 to maintain an internal sterile condition prior to use and after re-insertion of the cartridge with the skin sample inside. Removal of the cover allows the cartridge to be installed on the driver assembly with no operator contact being required. Re-installation of the cartridge into the package also will not require any operator contact except for replacement of the package cover.

### Removal of Sample from Cartridge

At the analysis laboratory, the sample may be removed from the cartridge as follows: The cartridge package 24 is opened either under sterile conditions when microbiological analysis is needed or under non-sterile conditions. Cartridge 1 is removed from the package by lifting it from its seat holding only the cartridge body 22. The skin sample is removed from the cartridge by holding the cartridge's central section over an appropriate receptacle and simultaneously rotating cam drive gear 21, which rotates the two cutters 18 to allow the skin sample to drop out of the cartridge into the laboratory receptacle. The cartridge and its package may then be discarded in an appropriate waste container.

### Performance Features

Performance features of this preferred embodiment include the following:

General:

Curved cut pattern for best closure and healing scenario.
Cut shape and size variable by using different cassettes.
Disposable cassette; clean, single use.
Single cut produces sample: 2 blades make tailored motion to achieve the desired cut shape.
Blades are retracted in cassette, extend automatically to start cut, stop at a central location at the end of the cut to trap sample.
Battery powered, no wires.
Integrated with stapler for efficient motion.
Rapid action: about 1 to 3 seconds to generate a sample
Staples and actuation system located in a disposable cartridge.
Option of adding a light source at the point of excision for improved illumination
   Sample Cartridge:
Cut shape best for staple closure, best healing configuration.
Sterile, single use.
Sealed in package, eject into sterile package container after sample is taken.
Multiple cut size and depth ranges for cassettes.
Snap in place on driver.
Metal or molded plastic cut blades.
Traps sample inside at end of cut
2 Blades cut simultaneously, guided in slots and by cams to rotate into skin to make the cut and capture the sample.
Lightweight, almost all components made from molded plastic.
Designed to protect driver unit and operator from blood contamination.
   Driver Unit:
Convenient size and shape for hand hold.
Battery inside.
Lightweight, mostly plastic construction.
Small dc gear motor drives cutters.
Small dc motor or gearmotor drives blades oscillation
Small dc gearmotor drives stapler
Small solenoids to retain and release the cartridge electromagnetically.
No contact with patient.
Clean, no lubrication required.
Light emitting diode optional
   Stapler Cartridge
Sterile, disposable, single patient use.
Sealed in package with sample cartridge.
Integral staple forming components.
Initially spread staples to pull wound edges together.
Lightweight, almost all components made from molded plastic.

### Staple Removal

Staples can be removed after the wound has healed using a prior art three prong staple removal tool. A two edge prongs slide under the top portion of the staple and the center prong is positioned over the top of the top portion of the staple. Downward pressure of the center prong causes the staple points to spread apart from each other and the legs of the staple to pull out of the skin.

While the present invention has been described above in terms of a preferred embodiment, persons skilled in the medical arts will recognize that many changes, alterations and additions can be made to the described device. For example, a simpler design could eliminate the back and forth oscillation of the blades and the medical stapler does not have to be incorporated in or attached to the driver, in fact as indicated the wound can be closed with stitches. While transparency of the cartridge is desirable, it is not absolutely necessary. Other obvious techniques are available for accurate placement of the cartridge on the skin. While battery power is desirable, wall power with an electrical connection is an obvious alternative and carries with it some obvious advantages, such as the unit could be lighter without the battery. Batteries used may be standard disposables or rechargeable types. The cartridge retention and release can be effected by a simple latch mechanism rather than through use of electromagnetic coils. The oscillation drive may be direct rather than via belt, but clear vision for proper placement of the sample cartridge on the patient may be compromised. There may be situations where users would prefer that the cartridge be reusable, such as to obtain several samples from the same person. Further, it should be stressed that the outer package of the driver device may be configured to fit ergonomically to the hand of the practitioner and these electronics and gears described herein would be placed into such a plastic form. Therefore, the reader should determine the scope of the invention from the appended claims and not the examples that have been given.

We claim:

1. A motor driven skin biopsy device comprising:
A) a drive unit comprising at least one drive motor,
B) electronic controls for controlling said drive motor,
C) at least one biopsy cartridge, each cartridge comprising two curved blades, and
D) a first set of linkage units linking said drive unit and said two blades and adapted to force said two blades, when driven by said drive unit, to cut into skin tissue to carve out a skin sample and to trap the sample with in the cartridge.

2. The device as in claim 1 wherein said at least one biopsy cartridge is a plurality of disposable cartridges.

3. The device as in claim 2 wherein said drive unit also comprises an oscillator driver motor, and oscillator linkages adapted to impart a back and forth oscillation of both of said two blades to facilitate cutting action of said blades.

4. The device as in claim 3 wherein said device also comprises a motor driven stapler unit for closing wounds left by the removal of skin samples.

5. The device as in claim 1 wherein said first set of linkage units is comprised of gears and cams.

6. The device as in claim 3 wherein said oscillator linkages are comprised of pulleys, gears and cams.

7. The device as in claim 4 wherein said motor driven stapler unit is driven by a stapler gear motor located in said drive unit.

8. The device as in claim 7 wherein said motor driven stapler unit is comprised of gears and cams.

9. The device as in claim 8 wherein said stapler unit is disposable

10. The device as in claim 2 wherein said plurality of disposable cartridges is a large number of disposable cartridges.

11. The device as in claim 1 wherein said at least one biopsy cartridge comprises slots to help guide at least some of the linkages in said first set of linkages.

12. The device as in claim 1 wherein a portion of said at least one cartridge is transparent to permit accurate location of said device over biopsy sample positions.

13. The device as in claim 12 wherein a portion of said drive unit is also transparent.

14. The device as in claim 1 wherein said blades and controls are adapted to obtain skin samples having the general shape of a wide canoe.

15. The device as in claim 1 wherein said drive unit is battery powered.

16. The device as in claim 1 wherein said drive unit is utility powered.

17. The device as in claim 1 wherein said device is adapted to excise skin samples in less than three seconds.

18. The device as in claim 1 and further comprising a container for each cartridge, said container being suitable for transport of said skin sample to a pathology laboratory.

19. The device as in claim 1 and further comprising sensors to limit the blades drive motor or gearmotor to less than one revolution via the electronic control unit.

20. The device as in claim 1 wherein said drive unit further comprises a trigger control button and cartridge release button.

21. The device as in claim 1 wherein said drive unit comprises a light source of light for illuminating precise surgical excision sites.

22. The device as in claim 1 wherein said drive unit is contained within an ergonomically designed plastic form for a better fit into a practioner's hand.
